(19) United States
(12) Patent Application Publication
(10) Pub. No.: US 2019/0125240 A1
(43) Pub. Date: May 2, 2019

(54) SELF-EVALUATION TOOL AND DIAGNOSTIC METHOD FOR HEALTHY AGEING

(71) Applicant: Jean-Pierre MICHEL, Geneva (CH)

(72) Inventor: Jean-Pierre MICHEL, Geneva (CH)

(21) Appl. No.: 16/141,058

(22) Filed: Sep. 25, 2018

Related U.S. Application Data
(60) Provisional application No. 62/577,806, filed on Oct. 27, 2017.

(51) Int. Cl. A61B 5/00; A61B 5/11; A61B 5/16; G16H 10/60; G16H 50/30; G16H 50/20

(52) U.S. Cl. CPC A61B 5/4088; A61B 5/4023; A61B 5/1124; A61B 5/743; A61B 2503/08; G16H 10/60; G16H 50/30; G16H 50/20; A61B 5/165

(57) ABSTRACT

A self-assessment tool, application program and method for assisting a person or medical doctor in diagnosing the person's nutritional, functional, physical and cognitive abilities in order to suggest remedial actions. The method includes displaying results that show each of the standard cognitive and physical abilities in a sized depiction with the patient's cognitive and physical abilities shown in another sized depiction such that when the patient's cognitive or physical abilities are lower than the standard abilities, the patient's sized depiction is smaller in size than the depiction of the standard abilities, and when the patient's cognitive or physical abilities are higher than the standard abilities, the patient's sized depiction is larger in size. This allows the patient's doctor to quickly and visually determine which abilities, if any, require remedial action to try to enhance or increase the patient's abilities to thus improve the patient's further aging and quality of life.

This application claims the benefit of U.S. provisional application No. 62/577,806 filed Oct. 27, 2017, the entire content of which is expressly incorporated herein by reference thereto.

### BACKGROUND

People living in the world today have the longest lifespans of any prior population. One of the challenges to population aging is to determine what abilities the aging person has that need attention or enhancement compared to other abilities that are in line with those of a person of a similar or younger age so that the person's quality of life is enhanced.

In general abilities of people in older age is reflected by their genetic inheritance as well as from the physical and social environments that the person experiences. Even so there are situations experienced by the person during his or her life travels that contribute to physical and mental deterioration causing the person to age and become dependent upon others more quickly that what would normally be expected. These deteriorations are sometimes not even discoverable until a time when treatments or remedial actions cannot be successfully undertaken.

The changes that constitute and influence aging are complex. At the biological level aging is associated with the accumulation of a wide variety of molecular and cellular damage. Over time this damage leads to a gradual decrease in physiological reserves, and increased risk of many diseases and a general decline in the intrinsic capacity of the individual. Ultimately, this damage leads to death of the person but these changes are neither linear nor consistent and they are only loosely associated with the person's age in years.

Regardless of the prior situations experienced by people during aging, there remains a need to promptly and effectively determine certain basic abilities of an aging person so that remedial actions can be taken in an effort to enhance or improve the person's daily functioning and quality of life during further aging. The earlier these abilities are understood, the earlier the remedial actions can be taken, and the more age-related disability can be delayed.

To frame how health and functioning might be considered an older age, a concept of intrinsic capacity is considered. This refers to the composite of all physical and mental capacities that an individual can draw on it any point in time. Another consideration is the environments in which the people and have it and their interactions with their environments. This combination of individuals and their environments as well as the interaction between them is termed the functional ability of the person. Accordingly, healthy aging is the process of developing and maintaining functional ability to enable well-being and intrinsic capacity in older ages.

Much of the preceding background information appears in the World Report on Ageing and Health published in 2015 by the World Health Organization (the “WHO Report”), a copy of which appears in the Appendix A annexed hereto and which is incorporated fully herein by reference thereto. The WHO Report notes that priorities for determining the capacities of persons are currently insufficient and that these need to be developed to include:
developing and reaching consensus on metrics, measurement strategies, instruments, tests and biomarkers for key concepts related to Healthy Ageing, including for functional ability, intrinsic capacity, subjective wellbeing, health abilities, personal abilities, genetic inheritance, multimorbidity and the need for services and care;
reaching consensus on approaches for assessing and interpreting trajectories of these metrics and measures during the life course. It will be important to demonstrate how the information generated can serve as inputs for policy, monitoring, evaluation, clinical or public-health decisions; and
developing and applying improved approaches for testing clinical interventions that take account of the different physiology of older people and multimorbidity.

Currently there are many existing scientifically accepted tests as well as non-scientific tests that could be provided or conducted on a patient to determine the patient's physical and mental abilities. This is known in general as a Comprehensive Geriatric Assessment (CGA). CGA was defined as a multidimensional, interdisciplinary diagnostic process focused on determining the medical, psychological, and functional capabilities of a frail elderly person in order to develop a coordinated and integrated plan for treatment and long-term follow-up. CGA differs from the standard medical evaluation by its concentration on frail older people with complex problems, emphasis on functional status and quality of life, use of interdisciplinary teams, and standardized and usually quantitative assessment scales (see A. Pilotto—Oxford Textbook of Geriatric Medicine 3rd Ed, J P Michel et al E D. Oxford University Press, Oxford, 2018).

There is a range of intensity of CGA, with screening assessments designed to identify older person's problems performed by primary care/community health workers, and diagnostic assessment and management of these problems carried out by a multidisciplinary team with geriatric training and experience (Rubenstein et al., 1991).

During the last 30 years, however, models of CGA have evolved in different healthcare settings to meet differing needs, becoming the foundation of ‘progressive’ geriatric care, including acute hospital care, day hospitals, rehabilitation units, nursing homes, and home visitation services (A Pilotto, supra).

First-generation CGA relies on batteries of single-domain measures, such as the Mini-Mental State Examination, Barthel Index, and Mini Nutritional Assessment. As a result, there exists no consistency or standardization of CGA batteries among different programs. This lack of standardization leads to provider and patient burden, inefficiencies in care that limited data with which to evaluate the quality of care. Second-generation CGA instruments include all geriatric domains and have been validated for specific settings (G Heckman et al, Oxford textbook of geriatric Medicine, 2018).

To achieve best outcomes for older people in their health and care, the concerns and priorities of older people themselves need to be more organized. It is best to have a standardized system for personalized assessment which can be applied to large populations of older people in primary care settings. In countries with well-developed specialist services more specialized or comprehensive assessment can be added (Philp et al WHO report XII 17).

CGA use resulted in the generation of various tools and tests for specific settings and diseases or conditions such as frailty, but the amount of existing material is overwhelming. In Pubmed there are today just under 40,000 published scientific papers in peer review journals on various test regimes, but there is no current way to ascertain how to use and apply this information to diagnose a person's intrinsic capacities and provide remedial actions to reduce ageing deterioration trajectories. And while a new generation of tools is primarily directed to determine frailty and medical deficiencies such as cancer, there currently are more than 70 different tools to detect frailty which again makes it difficult to assess which tests to use.

While of course detailed examinations of all of the abilities and capacities of the person can be undertaken by a physician, this requires significant time and expense to form a complete determination of the person's physical and mental abilities in order to diagnose an appropriate course of action for providing future improvement. For that reason, physicians are never doing CGAs. They always delegate these tasks to nurses, dietitians, social workers, and other supporting personnel which can provide a multidisciplinary team. Even so, it would take between 45 to 60 minutes for an appropriate examination, and this is not done by physicians because it is too long and too costly. Furthermore, only specialists working in geriatric departments have the correct personnel and team to do appropriate evaluations.

Organ specialists and surgeons know more and more that this CGA can allow them to avoid operating on patients who will not survive or have very severe complications, but despite this they do not know how to practice CGA. And as medical doctors are relatively busy in carrying out their role in treating patients for various maladies and injuries, there generally is not enough time for the physician or his medical personnel to conduct CGA, especially for persons in more or less generally good health but who may be experiencing or starting to experience aging issues. Thus, there is a need for improvement in the diagnosis of ageing in an elderly patient.

Accordingly, the present invention has been designed to meet this need to provide a relatively comprehensive but easy to implement method and diagnostic tool to assist doctors in promptly identifying aging abilities that are in need of improvement in order to promote healthy aging in the patient.

### SUMMARY OF THE INVENTION

The present invention relates to a method for assisting a medical doctor in efficiently and effectively diagnosing an aging patient's cognitive and physical status in order to determine whether remedial actions are necessary. This method includes providing the medical doctor with results from a self-assessment tool that obtains information of the patient's cognitive, physical and functional abilities. The method compares the obtained information with standard or average cognitive, physical and functional abilities based on a healthy person of the same age or age range as the patient. The results are displayed to show each of the standard or average cognitive, physical and functional abilities in a sized depiction with the patient's cognitive, physical and functional abilities shown in another sized depiction such that when the patient's cognitive, physical and functional abilities are lower than the standard or average abilities, the patient's sized depiction is smaller in size than the depiction of the standard or average abilities, and when the patient's cognitive, physical and functional abilities are higher than the standard or average abilities, the patient's sized depiction is larger in size than the depiction of the standard or average abilities. The method thus allows the doctor to quickly and visually diagnose and determine whether any of the patient's abilities require remedial action to try to enhance or increase the patient's cognitive, physical and functional abilities to standard or average abilities for a person of similar age to thus improve the patient's further aging.

Another embodiment of the invention is a system for determining cognitive, physical and functional abilities of a person. This system comprises a computer device running at least an application program that receives data from tests conducted upon or in conjunction with the device and that analyzes and scores the data, and a database for storing at least some of the data and scored test results. The application program is advantageously configured to allow a user to conduct a self-determination of his or her cognitive, physical and functional abilities. To do this, the application program includes instructions for obtaining information by the patient's self-determination of cognitive, physical and functional abilities from various tests that are conducted while using the computer device. These tests can include any combination of those mentioned in the detailed description that are used to measure physical, cognitive, nutritional, physiological, and/or quality of life abilities.

Yet another embodiment relates to a presentation slide creation system that automatically generates presentation slides. This system includes a slide creation application implemented on a computer device using computer-readable software instructions stored in non-transient memory. The slide creation application is configured to perform computer-implemented steps comprising conducting tests to obtain data of cognitive, physical and functional abilities of a user wherein the tests are conducted while using the computer device and by interacting with the device or by operating its touch screen, microphone, operational buttons or other interactive features.

Another aspect of the present invention relates to a self-assessment diagnostic tool comprising the computer system or presentation slide creation system disclosed herein. These systems are used to obtain the user's test results of cognitive and physical abilities and which includes: cognitive, physical and functional abilities of a healthy person of the same age or age range as the user, a comparison of the patient's and healthy person's abilities, and a display of the comparison results to illustrate whether the patient's cognitive, physical and functional abilities are higher or lower than the abilities of a healthy person.

The tool is preferably a mobile phone, electronic tablet or electronic watch and the user's test results can be and preferably are forwarded or presented to a medical doctor for diagnosis, analysis and determination of remedial actions that would typically include exercise when the patient's physical abilities are lower than that of a healthy person, or brain games or stress reducing activities when the patient's cognitive abilities are lower than that of the healthy person. Advantageously, the standard or average cognitive, physical and functional abilities are based upon data from healthy subjects determined by age groupings that include the age of the patient. The data can also be obtained from databases or other sources of information that attribute standard or average abilities for a person of a particular age or in an age range.

The patient's cognitive, physical and functional abilities are calculated from data obtained by the patient self-conducting certain physical and cognitive tests that are scientifically recognized as having value in evaluating the patient's intrinsic capacities. Preferred physical tests to be conducted include one, two or all three of the following:

a robustness test (5 self-reported evaluations); and/or

a balance test (one leg stand, both size, open eye); and/or

a flexibility test (for lower limb strength);

while preferred cognitive tests include one, two or all three of the following:

anxiety, stress and memory complaints (memory complaints testified by a proxy); and/or

a semantic memory evaluation; and/or

a visual retention test.

The cognitive, physical and functional abilities are visually illustrated by appropriately sized elements that can be represented by lines, bars, and solid or outlined circles, polygons or other shapes including a flower-type configuration wherein each flower petal represents a different standard or average ability and the patient's cognitive, physical and functional abilities are represented by smaller or larger size flower petals depending upon whether the patient's abilities are lower or greater than the standard or average abilities. Some of the petals illustrate the results of the physical tests while other petals illustrate the results of the cognitive tests. The results are immediately calculated and provided to the user.

As noted, the patient's abilities are preferably obtained by conducting self-assessment tests on a mobile or hand held electronic device with the results are displayed on a screen of an electronic device or if desired on a separate monitor or a printout. The electronic device is typically a mobile phone, electronic tablet or electronic watch. The invention also provides a visual comparison of the user's test results and cognitive, physical and functional abilities based on a healthy person of an age range that includes the age of the user, with the results immediately calculated and provided on the display to illustrate whether the patient's abilities are higher or lower than the abilities of a healthy person of an age range that encompasses the user's age. The test results can also indicate whether the person is at risk for losing certain abilities or whether certain abilities are starting to decline.

The displayed results may include the abilities of the healthy person in a sized depiction with the patient's cognitive, physical and functional abilities shown in another sized depiction such that when the patient's cognitive, physical and functional abilities are lower than the standard or average abilities, the patient's sized depiction is smaller in size than the depiction of the standard or average abilities, and when the patient's cognitive, physical and functional abilities are higher than the standard or average abilities, the patient's sized depiction is larger in size than the depiction of the standard or average abilities.

The invention also provides remedial actions of physical exercise when the patient's physical or functional abilities are lower than that of a healthy person in an age range that includes the age of the patient or providing remedial actions of brain games or stress reducing activities when the patient's cognitive or functional abilities are lower than that of a healthy person in an age range that includes the age of the patient. These can be provided by a physician that reviews the results or by the application program of the electronic device itself that scores and immediately provides the test results and relative depictions. Furthermore, the results can be evaluated quickly to provide remedial actions, such as in 10 to 15 minutes during a routine examination where the physician reviews the results of the self-assessment by the patient including the health/physical/cognitive/functional domains that are shown along with the patient's indication of smoking (or not) and his or her perceived quality of life. This optimizes the patient/physician interaction time and also allows the patient more time to use the tool to self-assess his or her abilities and obtain more accurate results than can be achieved in a rushed setting such as the physician's office.

### BRIEF DESCRIPTION OF THE DRAWINGS

These and other aspects of the present invention will be more readily apparent to those skilled in the art from a review of the description of the preferred embodiments which appear herein and which are illustrated by the associated drawings, wherein:

FIG. 1 is a schematic illustration of the self-evaluation tests that can be conducted to assess the person's cognitive, physical and functional abilities;

FIG. 2 is an illustration of a screen shot that shows a flower having petals of a predetermined size that represent normal, standard or average test results for a person in the age range that includes the age of the patient;

FIG. 3 is an illustration of a screen shot that shows one of the cognitive tests;

FIG. 4 is an illustration of a screen shot that shows the test results for a 50 year old person who is a business manager;

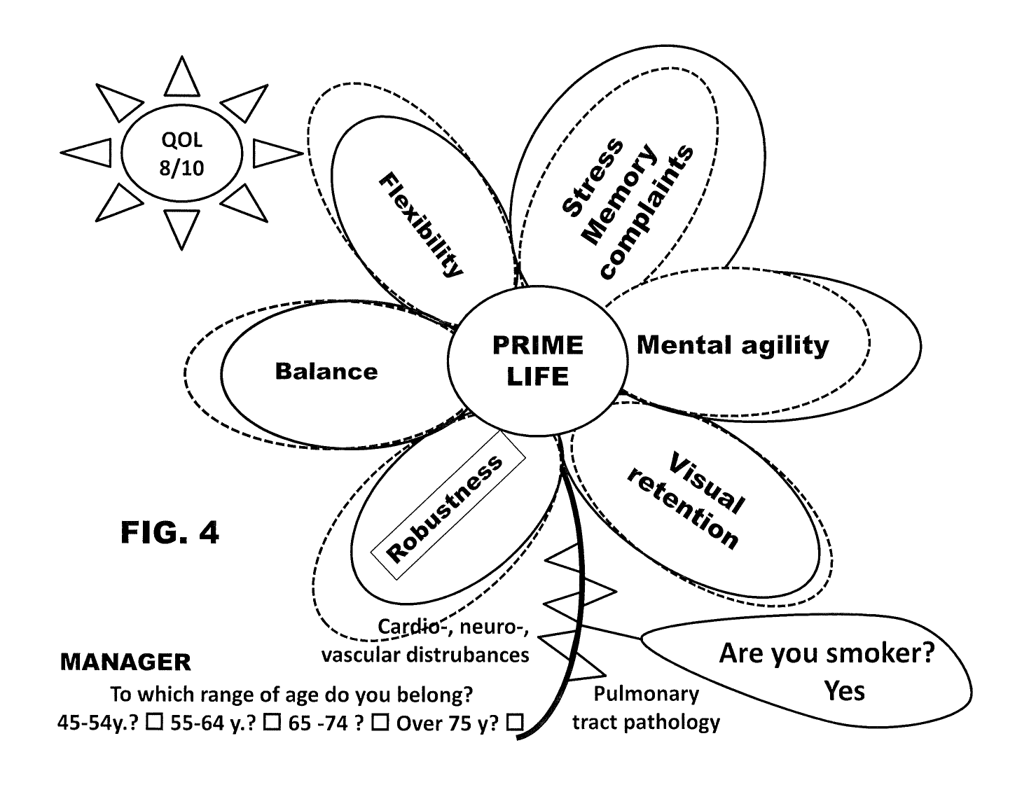

FIG. 5 is an illustration of a screen shot that shows the test results for a 66 year old person who is a concierge;

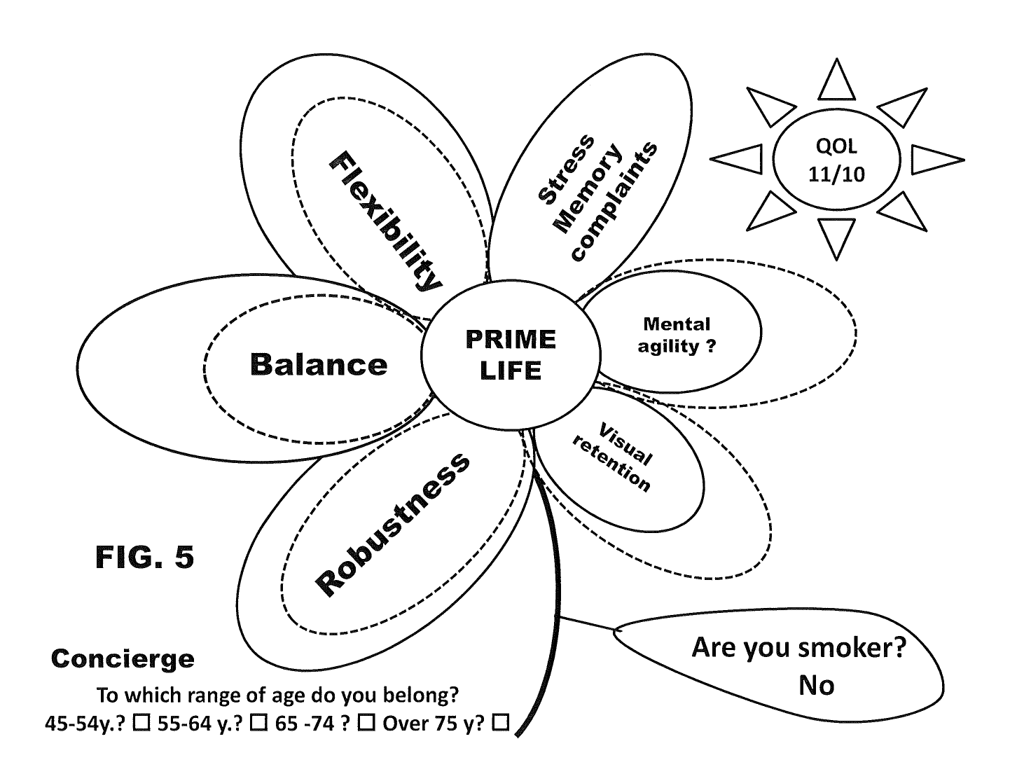

FIG. 6 is an illustration of a screen shot that shows the test results for a 77 year old person who is a general practitioner medical doctor;

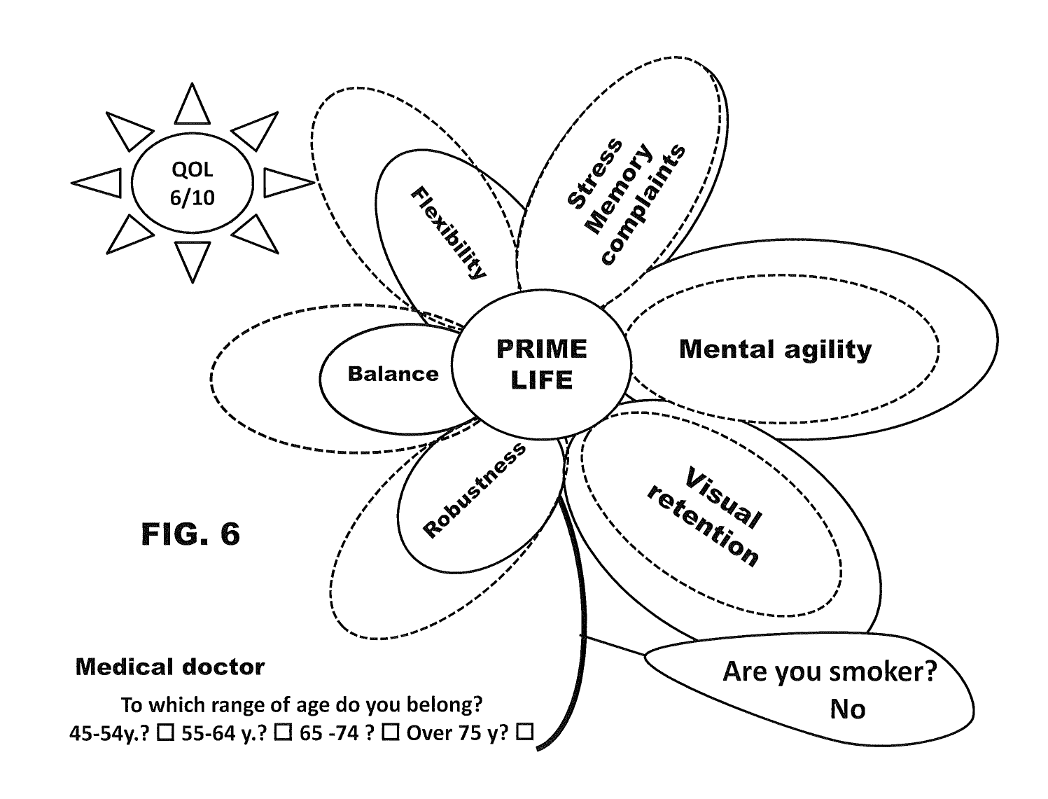

FIG. 7 is an illustration of the risks of poor performance in the tested categories;

FIG. 8 illustrates the scoring for the person's responses to the robustness evaluation;

FIG. 9 illustrates the scoring of the results for balance testing;

FIG. 10 illustrates the scoring for dual task testing of the person's balance;

FIG. 11 illustrates the scoring of the flexibility test measured by standing up from a chair;

FIG. 12 illustrates the questions utilized for a psychological evaluation of the patient;

FIG. 13 illustrates the scoring of the mental agility test;

FIG. 14 illustrates a visual retention test that is used in connection with the screenshot of FIG. 3; and

FIG. 15 illustrates how the participant can include his or her quality of life rating.

### DETAILED DESCRIPTION OF THE INVENTION

The present invention provides a self-evaluation tool in the form of a game or app that a person can review and conduct to provide important data regarding the person's physical and cognitive capabilities to determine how the person is ageing. This tool is generally referred to as Age Advisor and includes a game or app called Prime of Life. The main goals of the Age Advisor is to favor functional independence, autonomy, and well-being of the person using the tool. The Age Advisor game or app evaluates and helps a person control his or her aging process by providing a candid assessment of the person's health report, nutritional status, muscular mass and function, frailty and risk, balance, and cognition, along with information on how to enhance any deficient areas. The Age Advisor also can suggest that the person share his or her results with a family member of physician to obtain assistance in determining how best to proceed.

A full assessment is provided based on personalized results and timing. The app also suggest a regular repetition of the main exercises periodically to measure improvement and in particular after the user adopts a new diet, practices physical/balance/cognitive exercises or receives new or different medical prescriptions, vitamins, such as vitamin D, oral nutritional supplements or the like, so that the user is able to control his or her own development and monitor improvement. The app also provides regular feedback of selected but important personal results from the designed tests, including encouragements and or alarms when necessary. And if desired, the results of the test can be shared with or provided to family members or the person's physician. These additional individuals typically provide encouragement or additional advice to assist the person in undertaking activities that will slow down deterioration trajectories and enhance the person's wellbeing and future quality of life.

Therefore, the use of the app helps create a health prevention network addressing certain deficiencies in the physical and cognitive capabilities of the person. It also provides targeted advice and interventions to assist the individual in improving certain abilities. It also can be used to prevent age related disabilities from occurring more quickly than necessary. Repeating the exercises proposed by the results of the tests increase the person's physical abilities which then results in the avoidance of falls or other frailty issues. And another preferred and unexpected benefit is that the results of the game or app can be used by physicians for a rapid and effective diagnosis of the patient's physical and mental abilities, as well as to train other healthcare professionals in determining the optimum treatments and future or further exercises for the patient.

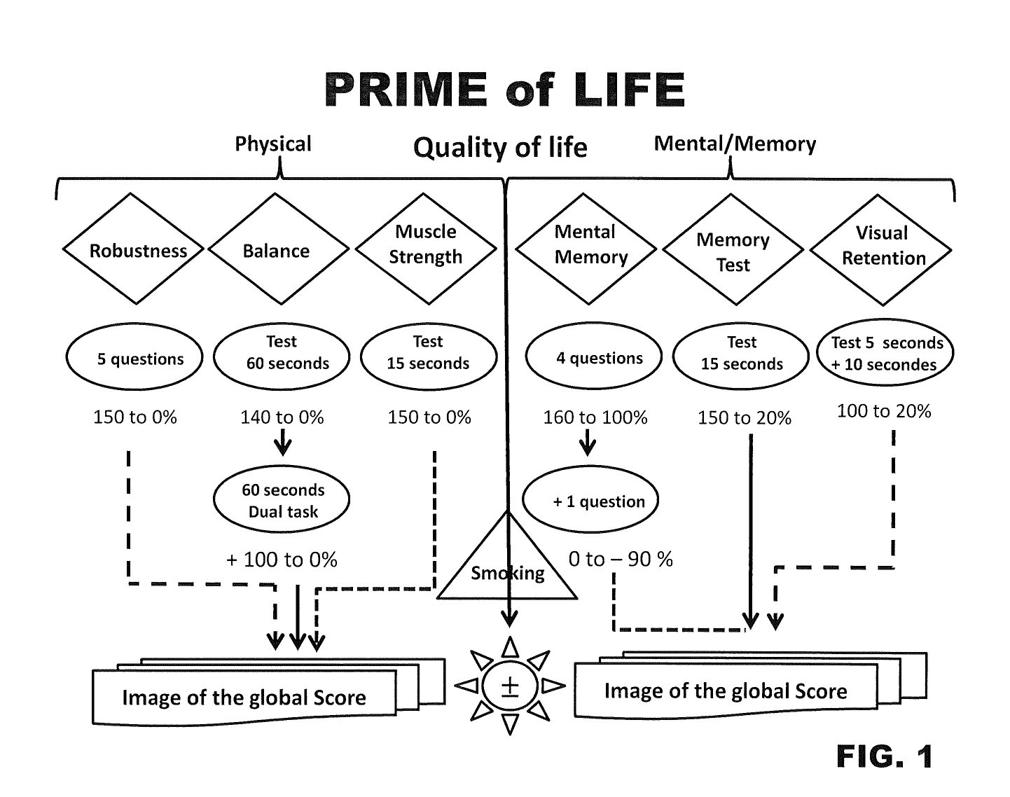

A schematic representation of a preferred arrangement of physical and cognitive tests appears in FIG. 1. In particular, the age advisor app presents a list of questions, physical tests and mental challenges to score the person's frailty, balance, physical capacities, and cognitive capabilities. The specific tests and measurements for physical abilities include nutrition screening, sarcopenia evaluation, normal gait speed and physiological reserve, while for cognitive testing various memory and knowledge tests are provided. These tests are scored and compared to standard or average results for a person of the same age range as the individual using the app.

The present invention provides the results in visual form to enable the physician to determine actions that assist the patient in building and maintaining intrinsic capacity as well as to enable greater functional ability in a person that has a given level of intrinsic capacity. This can be combined with additional intrinsic capacity increasing actions such as reducing risks of unfavorable activities such as avoiding smoking or being exposed to high levels of air pollution, encouraging other healthy behaviors such as physical or mental exercise activities, as well as to removing barriers that provide further risks to the patient such as avoiding high crime rate areas or dangerous traffic situations while also assisting the patient by providing services that foster capacity such as access to high quality foods and available health care. All of these activities can be combined as noted to increase intrinsic capacity and provide greater functional ability to influence healthy aging in the patient.

And an additional advantage of the present invention is that it allows people to grow into advanced older ages in better condition thus enabling such people to lead longer and healthier lives which in addition to enhancing their existence would also result in a reduction of healthcare costs.

The present invention preferably provides a self-assessment tool using available cell phone, electronic tablet or smart watch technologies, which tool is focused on an ageing individual from 45 to 75 or 85 years of age or older. The tool allows the individual to provide data by in effect playing a game (including answering the questions and short tests) in order to inform the user or their physician of the existing physical and cognitive abilities of the individual so that the physician can provide advice at midlife for enhancing the individual's intrinsic capacity and help avoid age-related disabilities and deterioration. The tool provides these results in a visual representation that can quickly and effectively provide the necessary information to assist the physician in determining remedial actions. And as the patient can play the game and obtain the information prior to visiting the physician, the time spent with the physician can be optimized and directed to determining the necessary care and further actions, rather than in collecting the information necessary to make those determinations.

In this specification, the terms “patient”, “person”, “user”, “subject” and “participant” are used interchangeably to refer to an aging person that is desirous of enhancing his or her physical or mental capacities to promote Healthy Ageing. In particular, the ageing person is one that has an age that is typically between 45 and 75 years.

As noted in the WHO Report, the definition of “Healthy Ageing,” is more than just the absence of disease but is instead defined as the process of developing and maintaining the functional ability that enables wellbeing in older age. The functional ability of the person is determined based on the intrinsic capacity of the patient so that the appropriate diagnosis and remedial actions can be proposed.

To conduct the self-examination to determine the intrinsic capacity of the patient, a number of tests were selected based on established scientific data, the inventor's long clinical experience as a geriatrician (over 30 years), and initial evaluations of individual tests performed with various volunteers. The specific tests that were chosen are considered to be complementary tests that are predictive of functional physical and cognitive abilities which typically decline with the aging of a person.

In a preferred embodiment, one or more of the following tests can be used in various combinations for an overall evaluation of the patient's physical and mental abilities:

1) Robustness vs. Frailty testing (Fried et al., J Gerontol A Biol Sci Med Sci. 2001; 56: M146-156);

2) Self-nutrition assessment (Huhmann et al., J Nutr Health Aging. 2013; 17: 339-344);

3) Muscle mass measurement (Rolland et al., J Am Geriatr Soc. 2003; 51(8):1120-1124);

4) Sarcopenia (Cruz-Jentoft et al, Age Ageing. 2010; 39: 412-423);

5) Balance measurements (Springer et al., J Geriatr Phys Therap. 2007; 30; 1-15);

6) Normal gait speed (Studenski et al., JAMA. 2011; 305: 55-58);

7) Physiological reserves (6 min fast gait speed test) (Depew, Respir Care. 2013 58: 2113-2119); and

8) Memory complaints and semantic memory (Amieva et al, Am Neurol Ass 2008; 64: 492-498).

All of these tests are well established and validated by the medical community. These tests are preferably carried out on a portable electronic device such as a mobile phone or tablet. The tests are integrated into the device by way of a software program or app that relies upon integrated device capabilities such as GPS, gyro, microphone (to receive words), touch interface, or other functionalities.

The present invention recognizes that a mobile app that resides on such devices could increase the scientific knowledge on the ageing process and better analyze the impact of possible interventions to lower the decline of ageing. The app is also predictive, preventive, personalized and participatory in order to effectively obtain the necessary results. The app also clarifies the constant interactions between the individual's abilities (or intrinsic capacity) and the person's own life surroundings (or functional abilities), as clearly stated in the WHO Report.

With the wider demographic use of i-phones and androids, a mobile app will definitely set the stage for a paradigm-shift from bio-medicine to functional medicine with deep societal impact. Engaging health care consumers as pioneers to use a mobile device able to globally assess their physical and cognitive performances will provide a collection of data which will increase insights into the ageing patho-physiology. This innovation will benefit the entire society (from users to patients) by decreasing age-related disability and care costs while favoring medical innovation and use of assistive devices. The invention is likely to be well accepted by the current younger population such as those people who will be 60+ in 2030 who are now over 45 and who along with younger people are current users of new communication technology such as the electronic phones, tablets, watches, glasses and other devices in current use and development.

In particular, the mobile app is predictive by providing information to determine an individual's risk of accelerating ageing decline, linking the silent and challenging physiological changes including: accumulation of years of life, stress, unhealthy life behavior/style or indeed trauma or disease. Ageing is a continuous process cumulating at midlife, with high physiological reserves and allowing good daily physical and cognitive abilities. This life period is also characterized by a great resilience, responsible of forgetting the insidious accumulation of various and multiple damage, which will be suddenly revealed by any life stressing event.

The app provides a great benefit which is to be able to inform an individual that his or her hectic work schedule and life activities are not accelerating his or her ageing speed. Knowing how to address deficiencies will help the person to adapt their personal, family and societal life conditions to stop neglecting their own health, limit their disproportionate ambition, and increase their wellness and ageing trajectories. To achieve this, the app also allows a personalized assessment, based on questionnaires (basic and more specific), as well as the performance of selected but very simple tests. Indeed, specific measures in daily life will be necessary to perfectly determine a few essential criteria focused primarily on physical and mental abilities.

Furthermore, the regular and long term repetition of measures at 1 or 3 months will pinpoint the early functional changes linked to sedentary habits, inadequate diet or transition to disease. Indeed, the dynamic evolution of the results is really the most important element of the personalized information, gaining fundamental insights into physiopathology. It demonstrates to the user if his/her current life habits, styles and behaviors intervene positively or not on his/her physical and mental capacities. It also indicates if such kinds of exercises have any positive effects. If not, alarms will be sent recommending to the individual to get a medical advice to better cope with the identified problem(s). The long term goal of the mobile app used from midlife is to identify personal risk factors, deliver advice and prevent age-related disability.

Another important feature of the mobile app is that it is participatory. After getting the global, but personalized results of the assessment, each individual could, if he/she wants, share them with a chosen proxy: spouse, partner, children or friends. A positive aspect of this mobile app is to be as amusing as a game you can play alone or with competitors. In this way, it will not only stimulate you to change your bad habit of eating, but also stop smoking, attend group exercises and conduct other beneficial activities. A variety of mental/cognitive stimulating activities, balance practice, weight lifting, bowling competitions, coordinative actions will inspired each individual using of the mobile app. The creation of network users would encourage everyone to live fully, active and enjoying discovering wellbeing until the latest years of life.

The mobile app thus promotes healthy ageing and allows personalized preventative interventions. Updated critical analysis of results of randomized controlled interventions, written in a simple language, will be available for the mobile app users who are facing one specific condition: physical exercise plus protein intake in case of loss of muscle mass/strength, Mediterranean diets for users with cardio-vascular issues, etc. In the near future it is envisaged to individualize answers to each user's health concerns or problems. Such a mobile app used by thousands of individuals in various world countries will allow setting up a large database of results, which dynamics will respond to essential questions of how it is possible to lower the functional decline linked to the ageing process. By this way, it will be possible to maintain good physiological reserve, increase physical functions, stimulate brain activities and prevent or delay the age-related disability. The impact of such positive outcomes will improve ageing/aged adults and caregivers well-being. Thus, the mobile app allows users, patients, researchers, physicians, and the entire health care community to join forces to transform the practice of medicine to make it more proactive than reactive—and, in turn, less expensive and more effective.

In particular, the data obtained during or after completion of the tests can be implemented at the software level on the applicable device. As examples, these include:

Whether the individual is a smoker or not.

Height and/or weight measurements (data entered on the touch screen);

Muscle strength (clicking on the device central button at different portions of the test such as when standing up, periodically when holding a position, etc. The software can select and store the best measures from any particular test or if the test is repeated multiple times, an average from the multiple tests);

Balancing (clicking on the device central button for starting each leg stance, for each of three trials, measuring duration if below 60 seconds, and playing sounds after the 60 second test time is reached to indicate that the test is complete). When the test was successful, a dual test is proposed with a high valued score;

Semantic memory (click on the device central button, for each word pronounced in 15 seconds; two trials are performed with the best score recorded). Concerning the words themselves, a glossary in different languages is provided to recognize whether the words correspond to the right semantic category;

The Isaacs Test, the same test proposed in a longer version (60 seconds) to detect preliminary signs of dementia;

Normal gait speed is measured in a 1 minute walk, which utilizes the GPS capabilities of the device. The measurement of gait speed is determined between 15 and 45 seconds after the start of the test to avoid abnormal walking at the start and the end of the test. A sound is emitted at the end of 60 seconds to indicate that the test is complete;

Physiological reserves: a 6 minute fast gait speed is measured. The distance (in feet or meters) will be calculated to correspond to the physiological reserve. GPS and Gyro capabilities are used for this test.

All of these features of a portable electronic device such as a mobile phone or tablet are essential in efficiently and effectively collecting the data for comparison to norms, standards or averages for the person's age and to determine where deficiencies or improvements are needed.

In a particularly preferred embodiment, the present invention provides an electronic game or app that is also a self-evaluation tool the core of which is an application program duly completed or amplified by an additional set of either questions or physical measurements which utilize algorithms for gathering, computing, and analyzing the various inputs (e.g., the replies to the questions and their related scores).

In the most preferred embodiment, six to eight domains are selected to illustrate or to qualify conveniently the health status of the ageing person, with each one of these domains provided in the form of a graphic representation. This representation can be depicted in a number of ways. While of course, a table can be used to compile the test results and compare it to standard or average values, this would take some time to review and determine the particular differences in the data. This is true even when colored text is used to assist in interpreting the data because this still requires a review of detailed information.

For these reasons, the present invention provides an illustration of the test results that are easily determinable visually in a manner that makes it quite easy and straightforward to identify and recognize the particular areas where a patient is deficient or requires further assistance. These visual representations can be made as lines, bars, and solid or outlined circles, polygons or other shapes with one length or size representing the norm, standard, average or 100% values and with the subject's data presented as a comparison (i.e., shorter or smaller or longer and larger) to the standard data. Colors, bolding or other contrasting features can be used to emphasize the results that show deficiencies or overachievement in the test results.

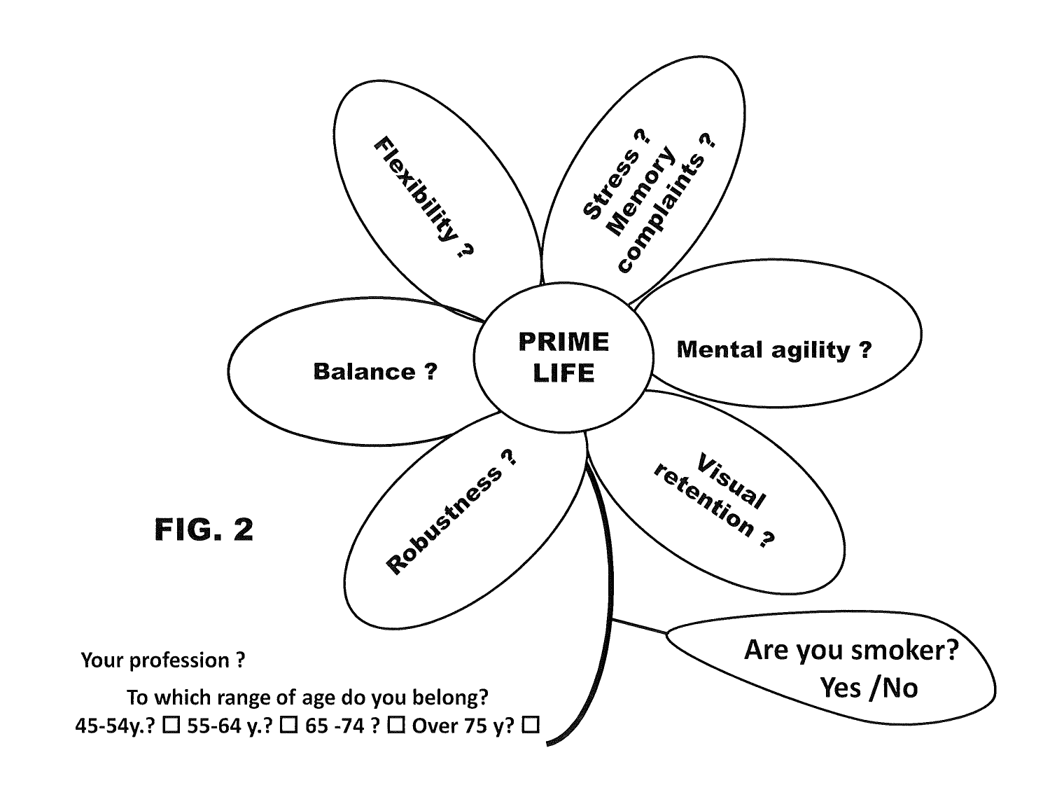

As shown in FIG. 2, a preferred representation is in the form of a flower having six petals initially provided with a petal surface or area that defines a score of 100% for the normal, standard or average values representing a good health status for a given age category and test. FIG. 2 also is a screen shot of one of the early screens of the app. The collected data then is analyzed and calculated to provide a different sized petal, with sizes that are decreased or smaller than those of the 100% scores representing abilities that require improvement and remediation. Conversely, sizes that are increased or larger than from those of the 100% scores represent areas where the person is above average in those abilities.

The tool initially asks the user to indicate his or her applicable age range. Although not included in the calculations, and as shown in FIG. 2, the tool also includes a question asking whether the individual is a smoker or non-smoker to provide additional relevant information to the physician although that particular indication is not scored or computed. An indication that the person is a smoker does cause the game or app to respond by pointing out the dangers of smoking and to also suggest ways to quit or reduce smoking in the patient.

The first three petals on the left side of the flower are questions and responses concerning the physical status of the subject whereas the three petals on the opposite side of the flower refer to the mental capacities of the subject.

Many of the questions are linked to and require a concrete physical interaction between the portable electronic device and the subject. These require, e.g., pressing buttons or entering data on the touch screen or providing audio answers to questions. The electronic device also processes this tactile or audio information to determine capabilities in performance or accuracy.

Following each set of questions for each test, a score is provided, the outcome of which is then read and stored by the software in view of its subsequent treatment by the algorithm.

At the end of the self-evaluation the subject is also questioned about his or her perception of the “quality of life”, an opinion of the subject which is simply stored for appearing on the final screen (results) but which is not computed further.

The graphic final representation of the complete self-evaluation is a superposition of both the initial 100% scores represented by one size of the flower petals and the measured scores from the self-evaluation which are represented in additional flower petals that are either larger, smaller or possibly the same size as the petals representing the 100% scores, with the differences when smaller than the standard indicating unbalanced health or functioning of the subject. The indication of quality of life provides additional information that may alert the physician as to whether the subject considers themselves to be ok or acceptable when the data may indicate potential upcoming deficiencies or issues.

As a further embodiment, the relative size differences between the 100% data and the patient's self-assessed abilities can represent the level of difference between the two. For example, a patient that achieves only half (50%) of the 100% values would have a petal that is half the size of the petal for the 100% value.

This self-evaluation tool and method can be repeated at periodical intervals, being then subject to iteration in order to show the evolution the health status of the subject, either as a whole or by reviewing each petal. The tests can become more complicated as the subject becomes familiar with the test with different wording or actions being required.

The invention utilizes software in the form of computer readable instructions that implement features or functionality described herein. The software is stored in non-volatile (non-transient) memory (e.g., programmable logic) and is executed by the electronic device which has a processor (computer processor) such as a smart phone, tablet or watch to provide the present features or functionality and related steps. The software is applied to the processor from non-volatile memory to provide the features or functionality. A combination of hardware and software implementation can be contemplated.

The interface of the device is software that is implemented and running on a computer using computer instructions that are stored in memory that, when executed by a processor, displays or provides the graphical and user-interactive features described herein (wherein the user can interact with the interface using an input device of the computer such as a keyboard, activation buttons, a microphone or the like). This includes the situation where the interface is provided through a web browser such as when a customer or analyst logs into the platform from their Internet browser running on their computer over an Internet connection to the platform. The interface is configured to implement the various related features described herein.

### Examples

The following example illustrates a particular app or electronic game that can be played or conducted on a mobile phone or tablet that allows a user to perform a self-evaluation of his or her physical and mental abilities. A number of categories are provided for completion by the user by either entering data or completing tests that allow the device to calculate the user's performance in the test. The results are shown in a visual display as a flower with 6 petals with three of the petals representing physical abilities and three others representing cognitive capabilities.

The app begins with a first screen that is a Welcome screen that identifies the app as the “Prime of Life” game.

Next a Table of Contents is provided that includes the following;

For the scoring of the tests, the following data is programmed into the app and used to calculate the results:

1^{st} Petal: Robustness (5 questions; score from 0 to 5)

| Status | Positive answers | Score |
|---|---|---|
| Very Robust | 0 | 100% |
| Robust | 1 | 80% |
| Pre-frail | 2 | 60% |
| Advanced Pre-frail status | 3 | 40% |
| Frail | 4 | 20% |
| Extremely Frail | 5 | 0% |

2^{nd} Petal: Balance
2^{nd} Petal: Simple balance test
The request is to stand on one leg (right and then left) open eyes, at least 60 seconds;
3 trials are possible on each side; the best result on each side is counted.
The score for each unilateral standing depends of the age of the participant:
For the first leg:

| First Leg | 60 sec | 50 sec | 40 sec | 30 sec | 20 sec | ≤10 sec |
|---|---|---|---|---|---|---|
| 45-54 y.o. | 80% | 60% | 40% | 30% | 10% | 0% |
| 55-64 y.o. | 100% | 80% | 60% | 40% | 20% | 0% |
| 65-74 y.o. | 120% | 100% | 80% | 60% | 40% | 10% |
| 75+ | 140% | 120% | 100% | 60% | 40% | 20% |

For the second leg:

| Second Leg | 60 sec | 50 sec | 40 sec | 30 sec | 20 sec | ≤10 sec |
|---|---|---|---|---|---|---|
| 45-54 y.o. | 80% | 60% | 40% | 30% | 10% | 0% |
| 55-64 y.o. | 100% | 80% | 60% | 40% | 20% | 0% |
| 65-74 y.o. | 120% | 100% | 80% | 60% | 40% | 10% |
| 75+ | 140% | 120% | 100% | 60% | 40% | 20% |

For scoring the petal add the best scores obtained on each leg and divide the results by 2

2^{nd} Petal: Dual-balance test
It is reserved to participants whose global score is above 80%; 3 trials are possible on each leg, always open eyes:

| Unilateral balance duration with dual test | | Size of the petal |
|---|---|---|
| <30 | sec | 120% |
| 30-39 | sec | 140% |
| 40-49 | sec | 160% |
| 50-59 | sec | 180% |
| 60-69 | sec | 200% |
| ≥60 | sec | 220% |

3^{rd} Petal - Physical test: Flexibility (lower limb strength)
Number of standings up from a chair

| | 150% | 120% | 100% | 50% | 20% | 0 |
|---|---|---|---|---|---|---|
| 45-54 y.o. | ≥18 | 17-15 | 14 | 13-12 | 11-9 | ≤8 |
| 55-64 y.o. | ≥16 | 15-13 | 12 | 11-9 | 8-7 | ≤6 |
| 65-74 y.o. | ≥14 | 13-11 | 10 | 9-8 | 7-6 | ≤5 |
| 75+ | ≥12 | 11-9 | 8 | 7-6 | 5-4 | ≤3 |

4^{th} Petal: Mental & memory evaluation

| Symptoms | Scores |
|---|---|
| NO stress, NO anxiety, NO memory complaint | 100% |
| STRESS: acute or chronic | +20% |
| Anxiety: acute or chronic | +20% |
| MEMORY: self-reported complaints | +20% |
| MEMORY: complaints testified by proxy | −90% |

5^{th} Petal: Mental agility (score of 10 words in 15 seconds)

| | 150% | 120% | 100% | 50% | 20% |
|---|---|---|---|---|---|
| Whatever your age | ≥14 | ≤13 ≥ 11 | 10 | ≥9 ≤ 7 | ≥6 |

Mental agility (score of 40 words in 60 seconds)

| | 150% | 120% | 100% | 50% | 20% |
|---|---|---|---|---|---|
| 45-54 y.o. | ≥40 | ≤40 ≥ 35 | ≥34 | ≤34 ≥ 28 | ≤28 |
| 55-64 y.o. | ≥40 | ≤40 ≥ 33 | ≥32 | ≤32 ≥ 26 | ≤26 |
| 65-74 y.o. | ≥40 | ≤40 ≥ 29 | ≥28 | ≤28 ≥ 24 | ≤24 |
| 75+ | ≥40 | ≤40 ≥ 25 | ≥24 | ≤24 ≥ 22 | ≤22 |

6^{th} Petal Visual retention (score max = 3/3)

| Recognition of shapes | Scores |
|---|---|
| 3/3 | 100% |
| 2/3 | 60% |
| 1/3 | 20% |

Quality of life (score from 0 to 10)

| Scoring | |
|---|---|
| ≥10 | Be careful, need to be interpreted in the personal context (previous scores) |
| 9-10 | Excellent |
| 7-8 | Good, but . . . important worries (have a look at the stress and anxiety scores) |
| 5-6 | Moderate (Need careful attention on physical capacities and anxiety) |
| ≤5 | Bad to very bad, to be interpreted in the personal context (previous scores) |

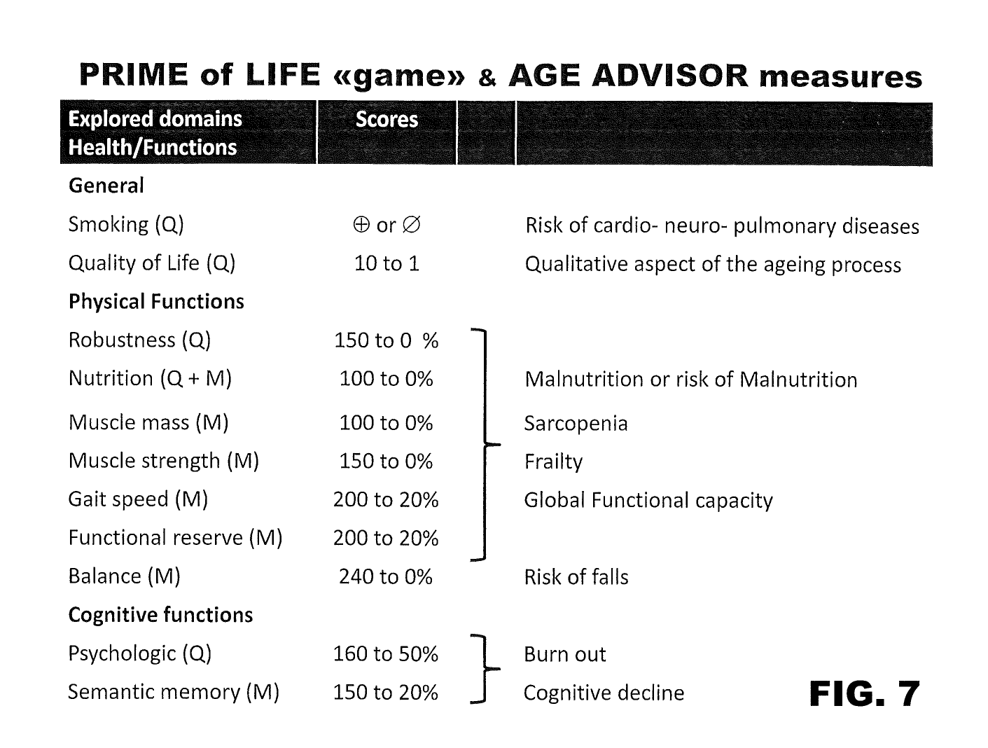

The results of the game and the risks of poor performance in the tested categories is shown in FIG. 7. The indication that the person is a smoker brings up the messages regarding the disease risks. Poor scores in physical functions indicate mound attrition, sarcopenia, frailty, or diminished global functional capacity and risks of falling. The cognitive function results can indicate burn out or general cognitive decline possibly leading to Alzheimer's disease.

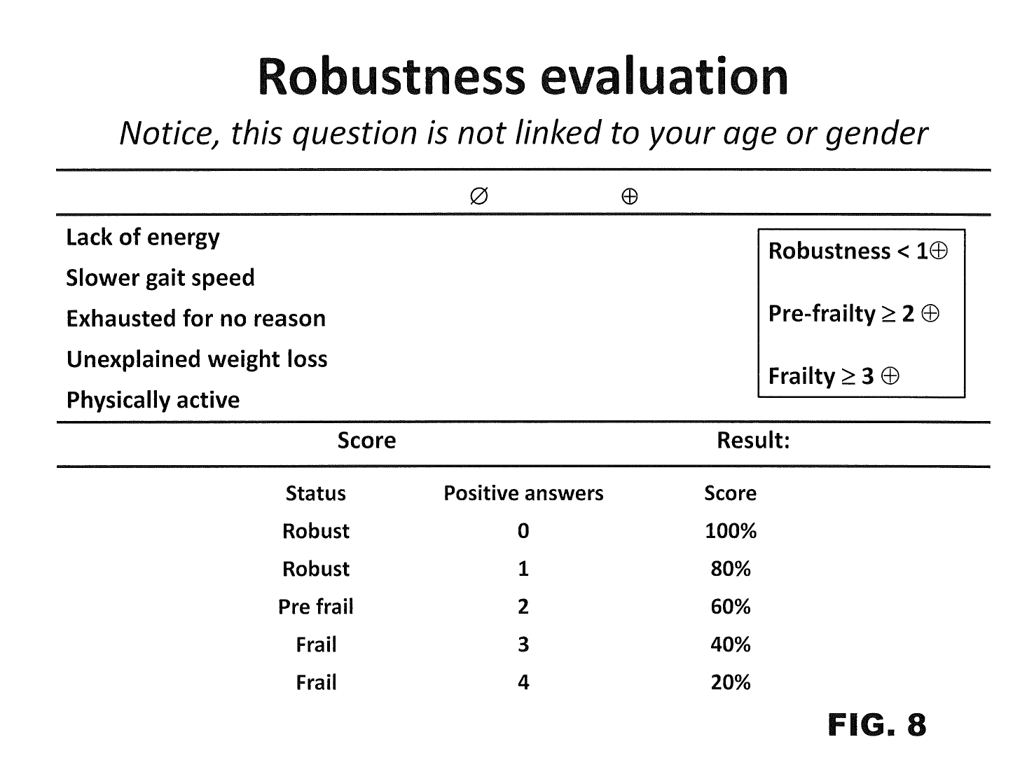

FIG. 8 illustrates the scoring for the person's responses to the robustness evaluation. Although this is an arbitrary score based on positive answers, a score of one or list indicates that the subject has sufficient robustness while higher scores indicate pre-frailty or frailty.

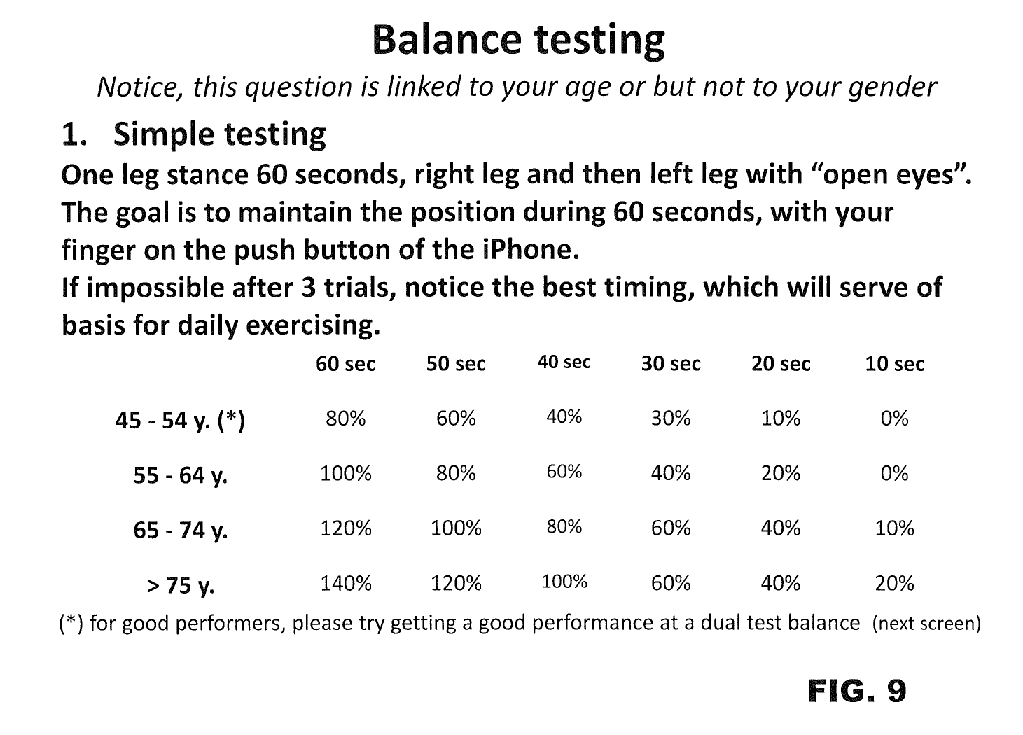

FIG. 9 illustrates the scoring of the results for balance testing with the longer times that the person can maintain standing position indicates there relative performance compared to others in the same age range.

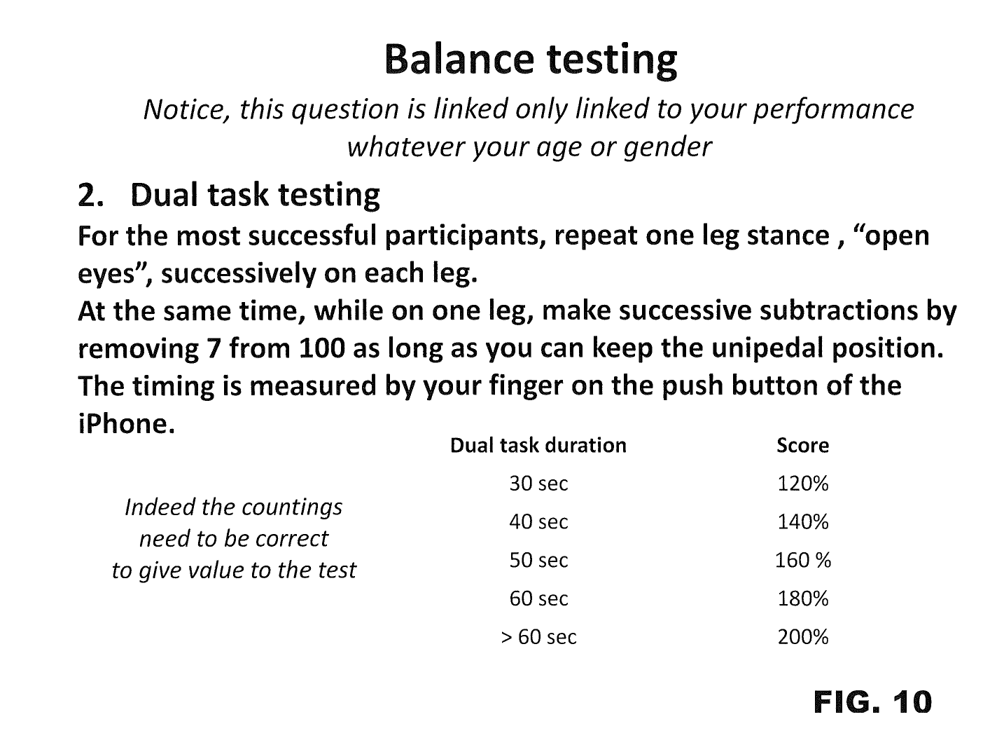

FIG. 10 illustrates the scoring for dual task testing of the person's balance with the results simply scored based on successful duration of the test.

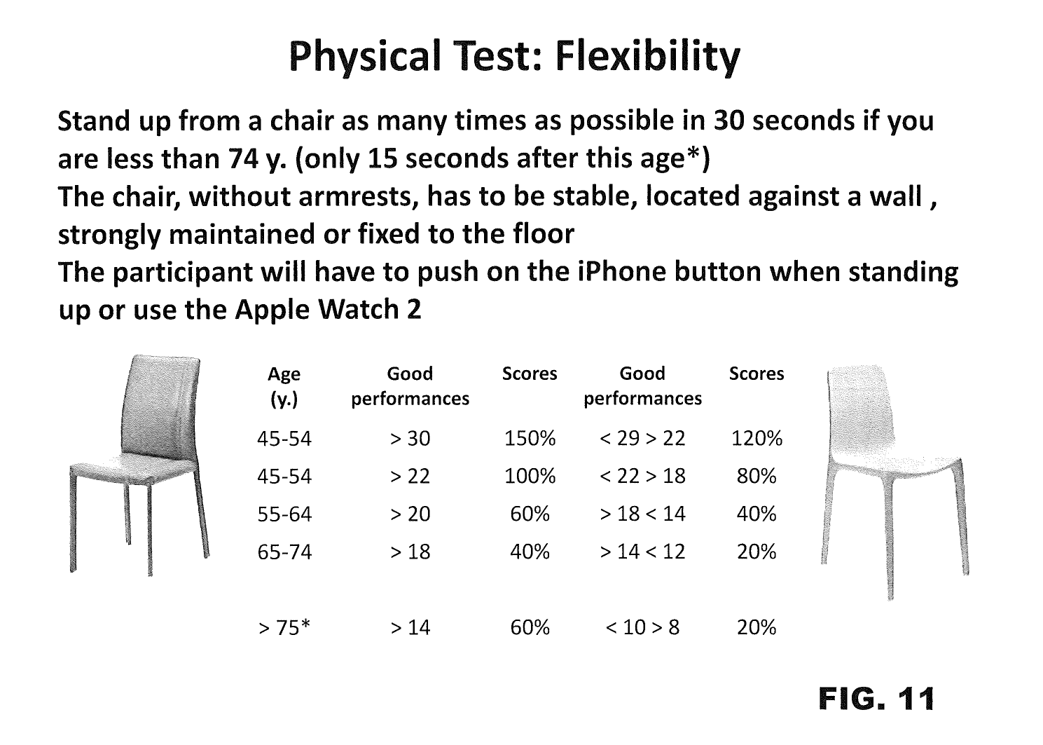

FIG. 11 illustrates the scoring of the flexibility test measured by standing up from a chair again with the scores indicating performance relative to the person's age group.

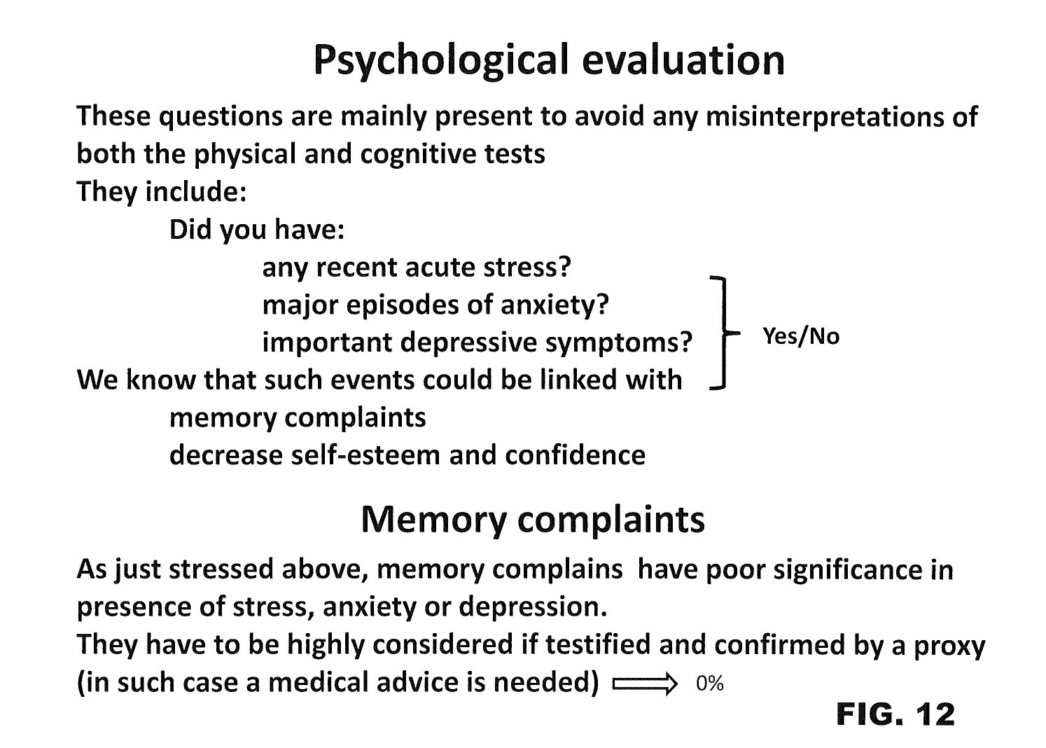

FIG. 12 illustrates the questions utilize for a psychological a valuation of the patient which is a predecessor of the evaluation of the patient's cognitive abilities.

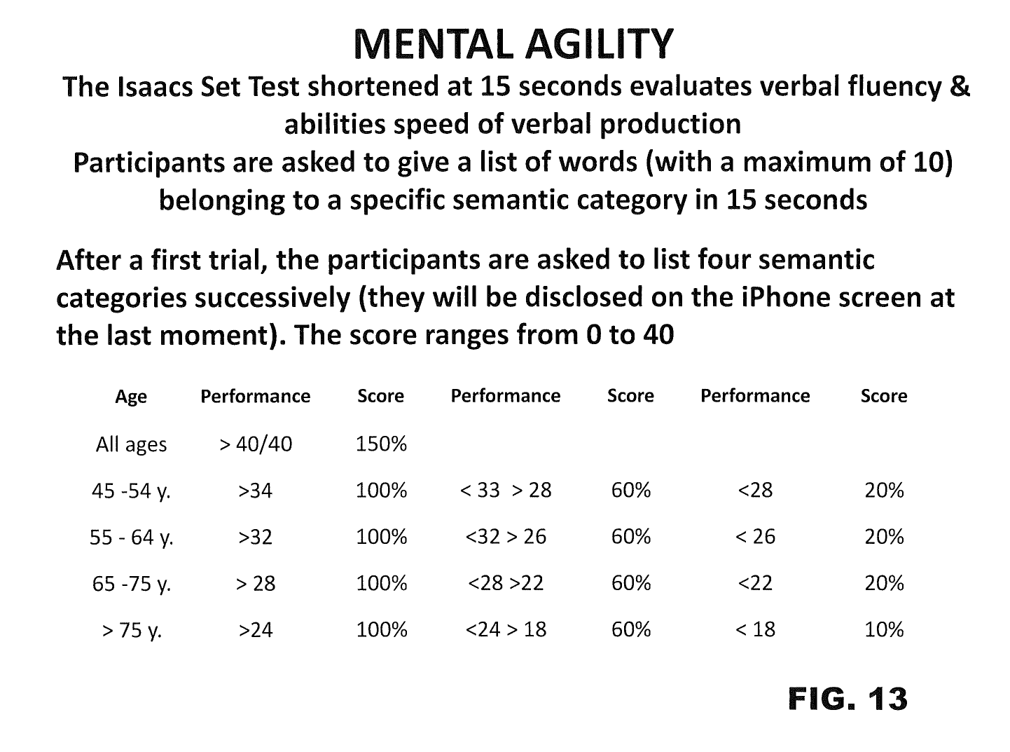

FIG. 13 illustrates the scoring of the mental agility test where in the participants are asked to prove five listings of certain words belonging to a specific category any particular time frame. The results are spoken into the electronic device which can also verify whether the answers are correct, for example asking for a listing of state or country capitals can be verified to assist in determining whether the correct answers are provided.

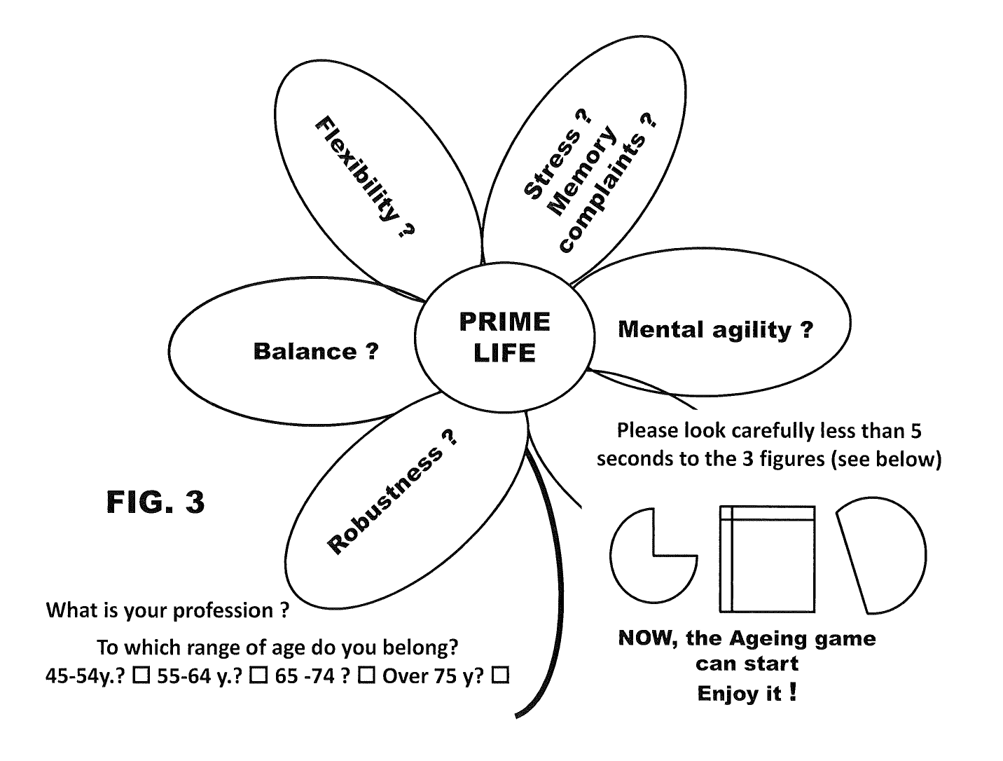

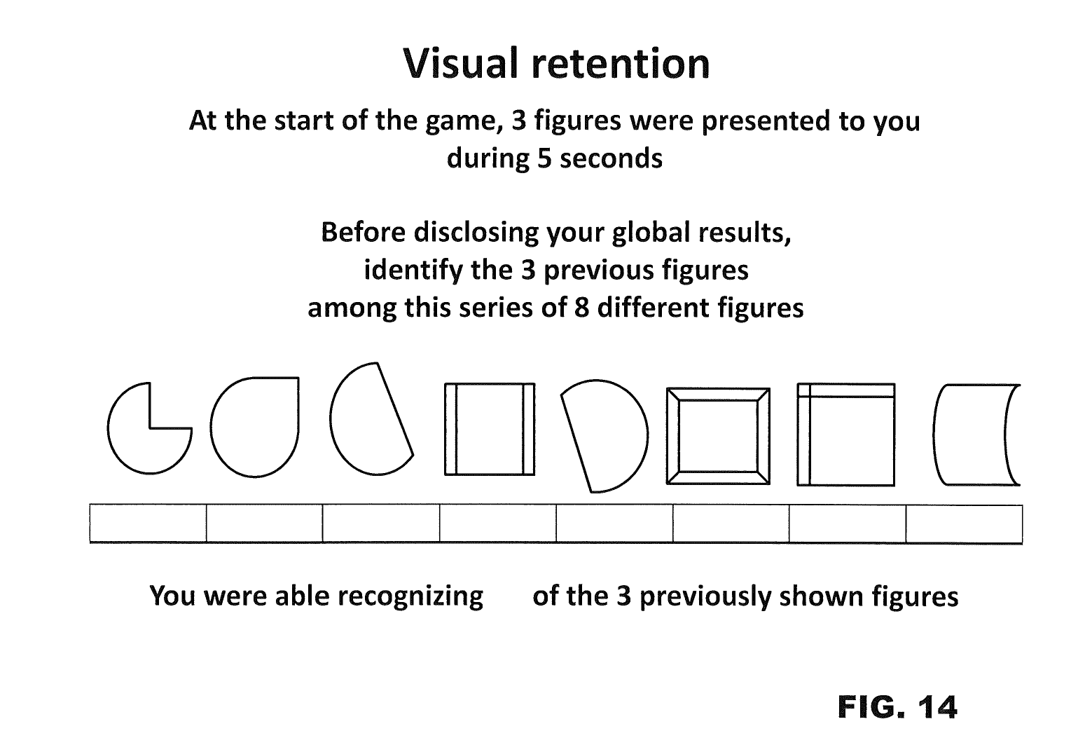

FIG. 14 illustrates a visual retention test that is used in connection with the screenshot of FIG. 3 where the patient was asked to remember certain figures. This screenshot illustrates that the user can check off which figures are remembered with the electronic device confirming the accuracy of the results.

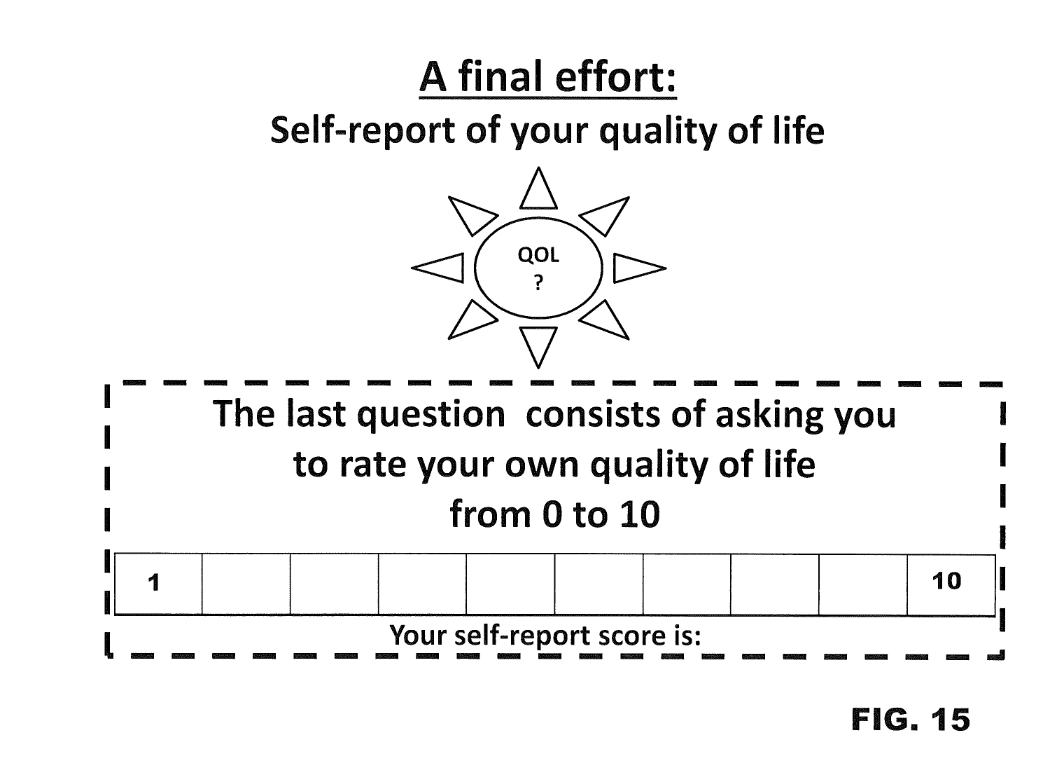

FIG. 15 illustrates how the participant can include his or her rating of their quality of life on a scale from 0 to 10.

These measured and calculated results illustrate various physical and cognitive abilities that are specific to the user's functionality, such as the user's nutrition, sarcopenia, walking ability (by measured gait speed and physiological reserve), and various memory abilities.

As noted the scoring and visual presentation of the results are designed to provide the individual and/or his physician to readily determine areas where the aging performance of the individual is behind what is considered to be the norm for his or her age grouping. This then leads to remedial actions to address those insufficiencies with the goal of improving the person's quality of life during further ageing.

The electronic device and application program can also become a tool for use by a physician in his or her office for pre-evaluating the patient before the patient is seen by the physician. This can be done well before an appointment with the doctor but it can also be conducted on the same day as the appointment because the electronic device and application program calculate the results immediately after completion of the tests to create the visual display of the results. Conducting the tests in the physician's office also enables other medical personnel to observe the patient and provide additional notes that may be helpful to the physician. During the appointment with the physician, the physician can review the displayed results to immediately understand the main problems of the patient.

The tool can also be used by surgeons or oncologists wherein the test results can provide information that the doctor can use to evaluate the risks involved in treating the patient and what to do before intervention to favor the outcome, e.g., to increase nutrition or administer additional gents that can assist the patient, or to require physical exercises to be conducted.

From the preceding, it is to be understood realized that the optimum visual results to be displayed can include variations and tolerances in size, shape, form, function and use, all of which are deemed readily apparent and obvious to the skilled artisan, such that all equivalent relationships to those illustrated in the drawings and described in the specification are intended to be encompassed by the claims appended hereto.

Unless defined otherwise, all technical and scientific terms used herein have same meaning as commonly understood by one of ordinary skill in the art to which this invention belongs. Also, as used herein and in the appended claims, the singular form “a”, “and”, and “the” include plural referents unless the context clearly dictates otherwise. All technical and scientific terms used herein have the same meaning.

The foregoing detailed description is considered as illustrative only of the principles of the invention. Further, since numerous modifications and changes will readily be apparent to those having ordinary skill in the art, it is not desired to limit the invention to the exact embodiments depicted and described. Accordingly, all suitable modifications and equivalents that may be resorted to are intended to be included within the scope of the appended claims.

What is claimed is:

1. A method for assisting a medical doctor in efficiently and effectively diagnosing an aging patient's cognitive and physical status in order to determine whether remedial actions are necessary, which comprises:
providing the medical doctor with results from a self-assessment tool that obtains information of the patient's cognitive, physical and functional abilities and that compares such information with standard or average cognitive, physical and functional abilities based on a healthy person of the same age or age range as the patient, and
displaying the results to show each of the standard or average cognitive, physical and functional abilities in a sized depiction with the patient's cognitive, physical and functional abilities shown in another sized depiction such that when the patient's cognitive, physical and functional abilities are lower than the standard or average abilities, the patient's sized depiction is smaller in size than the depiction of the standard or average abilities, and when the patient's cognitive, physical and functional abilities are higher than the standard or average abilities, the patient's sized depiction is larger in size than the depiction of the standard or average abilities,
thus allowing the doctor to quickly and visually diagnose and determine whether any of the patient's abilities require remedial action to try to enhance or increase the patient's cognitive, physical and functional abilities to standard or average abilities for a person of similar age to thus improve the patient's further aging.

2. The method of claim 1 wherein the standard or average abilities are based upon data from healthy subjects determined by age groupings that include the age of the patient, and the patient's cognitive, physical and functional abilities are calculated from data obtained by the patient self-conducting certain physical and cognitive tests that are scientifically recognized as having value in evaluating the patient's intrinsic capacities.

3. The method of claim 2 wherein the physical tests include one, two or all three of the following:
a robustness test (5 self-reported evaluations); and/or
a balance test (one leg stand, both size, open eye); and/or
a flexibility test (for lower limb strength); and
wherein the cognitive tests include one, two or all three of the following:
anxiety, stress and memory complaints (memory complaints testified by a proxy); and/or
a semantic memory evaluation; and/or
a visual retention test.

4. The method of claim 1 wherein the sizes of the patient's cognitive, physical and functional abilities and the standard or average abilities can be represented by lines, bars, and solid or outlined circles, polygons or other shapes including a flower-type configuration wherein each flower petal represents a different ability and the patient's abilities are represented by smaller or larger size flower petals depending upon whether the patient's abilities are lower or greater than the standard or average abilities with some of the petals illustrating the results of the physical tests while other petals illustrate the results of the cognitive tests.

5. The method of claim 1 wherein the patient's abilities are self-obtained by conducting the tests on a mobile or hand held electronic device and the results are displayed on a screen of an electronic device, an associated monitor, or on a printout.

6. The method of claim 1, which further comprises providing remedial actions of exercise when the patient's physical abilities are lower than that of a healthy person in an age range that includes the age of the patient or providing remedial actions of brain games or stress reducing activities when the patient's cognitive abilities are lower than that of a healthy person in an age range that includes the age of the patient.

7. A system for determining cognitive, physical and functional abilities of a person, comprising:
a computer device running at least an application program that receives data from tests conducted upon or in conjunction with the device and that analyzes and scores the data, and a database for storing at least some of the data and scored test results, wherein the application program is configured to allow a user to conduct a self-determination of his or her cognitive, physical and functional abilities, and includes:
instructions for obtaining information by the patient's self-determination of cognitive, physical and functional abilities from various tests that are conducted while using the computer device, wherein the physical abilities include one, two or all three of the following which are conducted by interacting with the device or by operating its touch screen or operational buttons:
a robustness test (5 self-reported evaluations); and/or
a balance test (one leg stand, both size, open eye); and/or
a flexibility test (for lower limb strength);
wherein the cognitive abilities include one, two or all three of the following tests conducted on the device:
answers provided to questions regarding anxiety, stress and memory complaints; and/or
answers provided to a semantic memory evaluation; and/or
answers provided to a visual retention test;
wherein the application program scores the test results; and
wherein the portable computer device includes a display that illustrates the user's test results that are scored by the application program.

8. The system of claim 7, wherein the computer device is a mobile phone, electronic tablet or electronic watch and the application program immediately calculates and provides a visual comparison of the user's test results and cognitive and physical abilities based on a healthy person of an age range that includes the age of the user, with the results provided on the display to illustrate whether the patient's abilities are higher or lower than the abilities of the healthy person of an age range that encompasses the user's age.

9. The system of claim 8, wherein the displayed results include the abilities of the healthy person in a sized depiction with the patient's cognitive and physical abilities shown in another sized depiction such that when the patient's cognitive or physical abilities are lower than the standard or average abilities, the patient's sized depiction is smaller in size than the depiction of the standard or average abilities, and when the patient's cognitive or physical abilities are higher than the standard or average abilities, the patient's sized depiction is larger in size than the depiction of the standard or average abilities.

10. The system of claim 9 wherein the sizes of the patient's cognitive, physical and functional abilities and the standard or average abilities are represented by lines, bars, and solid or outlined circles, polygons or other shapes including a flower-type configuration wherein each flower petal represents a different ability and the patient's abilities are represented by smaller or larger size flower petals depending upon whether the patient's abilities are lower or greater than the standard or average abilities with some of the petals illustrating the results of the physical tests while other petals illustrate the results of the cognitive tests.

11. The system of claim 7, wherein the patient's abilities are obtained by conducting the tests on the computer device that includes the application program and the results are displayed on a screen of the device, on a separate monitor, or on a page that is printed from the results shown on the device.

12. The system of claim 7, wherein the application program includes providing remedial actions of exercise when the patient's physical abilities are lower than that of a healthy person in an age range that includes the age of the patient or providing remedial actions of brain games or stress reducing activities when the patient's cognitive abilities are lower than that of a healthy person in an age range that includes the age of the patient.

13. A presentation slide creation system that automatically generates presentation slides, comprising:
a slide creation application implemented on a computer device using computer-readable software instructions stored in non-transient memory, wherein the slide creation application is configured to perform computer-implemented steps comprising:
conducting tests to obtain data of cognitive and physical abilities of a user wherein the tests are self-conducted by the user on the computer device, wherein the physical abilities include one, two or all three of the following which are conducted by interacting with the device or by operating its touch screen or operational buttons:
a robustness test (5 self-reported evaluations); and/or
a balance test (one leg stand, both size, open eye); and/or
a flexibility test (for lower limb strength);
wherein the cognitive abilities include one, two or all three of the following tests conducted on the device:
answers provided to questions regarding anxiety, stress and memory complaints; and/or
answers provided to a semantic memory evaluation; and/or
answers provided to a visual retention test;
scoring the test results to provide values that indicate the cognitive and physical abilities of the user; and
displaying at least the user's test results that are scored.

14. The system of claim 13, wherein the computer device is a mobile phone, electronic tablet or electronic watch and the slide creation application is configured to perform additional computer-implemented steps comprising:
providing a visual comparison of the user's test results with cognitive and physical abilities based on a healthy person of an age range that includes the age of the user, with the results displayed to illustrate whether the patient's cognitive, physical and functional abilities are higher or lower than the abilities of the healthy person of an age range that encompasses the user's age.

15. The system of claim 14, wherein the displayed results include the abilities of the healthy person in a sized depiction with the patient's cognitive, physical and functional abilities shown in another sized depiction such that when the patient's cognitive, physical and functional abilities are lower than the standard or average abilities, the patient's sized depiction is smaller in size than the depiction of the standard or average abilities, and when the patient's cognitive, physical and functional abilities are higher than the standard or average abilities, the patient's sized depiction is larger in size than the depiction of the standard or average abilities.

16. The system of claim 15, wherein the sizes of the patient's cognitive, physical and functional abilities and the standard or average abilities are represented by lines, bars, and solid or outlined circles, polygons or other shapes including a flower-type configuration wherein each flower petal represents a different ability and the patient's abilities are represented by smaller or larger size flower petals depending upon whether the patient's cognitive, physical and functional abilities are lower or greater than the standard or average abilities with some of the petals illustrating the results of the physical tests while other petals illustrate the results of the cognitive tests.

17. The system of claim 13, wherein the slide creation application is configured to perform additional computer-implemented steps comprising:
conducting the tests on the computer device that includes the application program in order to obtain the patient's abilities, and
displaying the results on a screen of the device or transferring the displayed results to a separate monitor, or to a printer that prints a page with the results.

18. The system of claim 13, wherein the slide creation application is configured to perform additional computer-implemented steps comprising:
providing remedial actions of exercise when the patient's physical abilities are lower than that of a healthy person in an age range that includes the age of the patient or providing remedial actions of brain games or stress reducing activities when the patient's cognitive abilities are lower than that of a healthy person in an age range that includes the age of the patient.

19. A self-assessment diagnostic tool comprising the computer system of claim 7 which has been used to obtain the user's test results of cognitive, physical and functional abilities and which includes:
cognitive, physical and functional abilities of a healthy person of the same age as the user,
a comparison of the patient's and healthy person's abilities, and
a display of the comparison results to illustrate whether the patient's abilities are higher or lower than the abilities of the healthy person.

20. The diagnostic tool of claim 19 wherein the computer device is a mobile phone, electronic tablet or electronic watch and wherein the user's test results are forwarded or presented to a medical doctor for analysis and a determination of: remedial actions of exercise when the patient's physical abilities are lower than that of a healthy person in an age range that includes the age of the patient, or remedial actions of brain games or stress reducing activities when the patient's cognitive abilities are lower than that of a healthy person in an age range that includes the age of the patient.

21. A self-assessment diagnostic tool comprising the computer system of claim 13 which has been used to obtain the user's test results of cognitive, physical and functional abilities and which includes:
cognitive, physical and functional abilities of a healthy person of the same age as the user,
a comparison of the patient's and healthy person's abilities, and
a display of the comparison results to illustrate whether the patient's abilities are higher or lower than the abilities of the healthy person.

22. The diagnostic tool of claim 21 wherein the computer device is a mobile phone, electronic tablet or electronic watch and wherein the user's test results are forwarded or presented to a medical doctor for analysis and a determination of: remedial actions of exercise when the patient's physical abilities are lower than that of a healthy person in an age range that includes the age of the patient, or remedial actions of brain games or stress reducing activities when the patient's cognitive abilities are lower than that of a healthy person in an age range that includes the age of the patient.
